(19) United States
(12) Patent Application Publication
(10) Pub. No.: US 2025/0025528 A1
(43) Pub. Date: Jan. 23, 2025

(54) MATERIALS AND METHODS FOR PREVENTING OR REDUCING TOXICITY TO ORGANOPHOSPHATES AND OTHER TOXIC MATERIALS

(71) Applicant: QUORUM INNOVATIONS, LLC, SARASOTA, FL (US)

(72) Inventors: NICHOLAS T. MONSUL, SARASOTA, FL (US); EVA A. BERKES, SARASOTA, FL (US); KAROL GLINIEWICZ, SARASOTA, FL (US); YU-HSIEN LIAO, SARASOTA, FL (US); I-FANG HSIN, SARASOTA, FL (US)

(21) Appl. No.: 18/707,357

(22) PCT Filed: Nov. 7, 2022
(86) PCT No.: PCT/US2022/079375

§ 371 Date: May 3, 2024

Related U.S. Application Data
(60) Provisional application No. 63/276,091, filed on Nov. 5, 2021.
(60) Provisional application No. 63/335,336, filed on Apr. 27, 2022.

(51) Int. Cl. A61K 38/16; A61K 35/00; A61K 35/747; G01N 33/50

(52) U.S. Cl. CPC A61K 38/164; A61K 35/747; G01N 33/5014; G01N 33/5088; A61K 2035/11

(57) ABSTRACT

The present invention provides materials and methods for alleviating organophosphate toxicity. More specifically, the invention provides pharmaceutical compositions comprising a protein, and/or biologically-active fragments and variants thereof, and methods of using the same.

### CROSS-REFERENCE TO RELATED APPLICATIONS

This application claims priority to U.S. Provisional Patent Application Ser. Nos. 63/276,091, filed Nov. 5, 2021; and 63/335,336, filed Apr. 27, 2022, each of which are incorporated herein by reference in their entireties.

### SEQUENCE LISTING

The Sequence Listing for this application is labeled “SeqList-as filed.xml,” which was created on Nov. 7, 2022, and is 8 KB. The Sequence Listing is incorporated herein by reference in its entirety.

### BACKGROUND OF THE INVENTION

Organophosphates (OP) are toxic compounds that pose a serious threat to human health worldwide, causing over 100,000 deaths annually that arise from hazardous accidental and occupational exposures (e.g., organophosphate pesticides), suicidal attempts, or due to weaponized OP agents used in terrorist attacks or for chemical warfare (CDC MMWR 1984, Lin et al., 2008; Meulenbelt and Nieuwenhiuzen, 2016; Mew et al., 2017; Bertolote et al., 2018; OPCW note 2020).

As potent acetylcholine esterase (AChE) inhibitors, organophosphates often block the enzyme quickly and irreversibly, leading to acetylcholine accumulation in neuromuscular synapses resulting in serious injury or death. Because of this quick action of AChE inhibitors and the rapid onset of symptoms, there is a very short time for victims of organophosphate poisoning to be diagnosed properly, receive antidotes and medical treatment (Hulse et al., 2019).

The duration of exposure to OPs can be assessed using biomarkers to determine the AChE levels. At low doses OP agents induce visual incapacitation. Topical OP ocular exposure leads to a reduction in cholinesterase activity in the iris and ciliary muscle with subsequent elevation of acetylcholine in the anterior chamber fluid of the eye. This cholinergic over-stimulation action on the pupillary sphincter results in a marked miosis with dimming of vision, decrease in spatial visual ability with paralysis of the ciliary body and decreased lens accommodation, and a decline in adapting to illumination changes due to desensitization of pupillary muscarinic receptors (Egoz 2017; Dabisch 2008; Genovese 2008; Lund-Karlsen and Fonneum 1976; Takayanagi 1993). In addition, the cholinergic over stimulation action induces ciliary muscle spasm which may lead to blurred vision and myopia (Cannard, 2006; Nohara and Segawa, 1996; Rengstorff 1985; Smith and Smith 1980; Yanagisawa, 2006).

The United States Environmental Protection Agency (EPA) banned most nonagricultural use of OPs in 2001. This is due to the toxic nature, societal health concerns, and environmental concerns associated with the use of OPs. Exposure to OP pesticides can occur through many pathways, such as food contamination, environmental and household pollution, proximity to agricultural fields, and agricultural work. Farmworkers in the United States have been one of the most susceptible groups to organophosphate exposure.

The use of OPs in chemical warfare was first documented by Gerhard Schrader in the 1930s. The first chemicals made were called G series and included Sarin, Tabun, and Soman. Later work was done that expanded on the Gerhard Schrader work and produced VX nerve agents, which were a magnitude more potent than the G series. When exposed to VX or G series toxins in the body's signaling mechanisms between the nervous and muscular systems become disrupted. This causes neuromuscular blockage, flaccid paralysis and death by asphyxiation.

Currently, the antidote options (e.g., oximes) and medical protocols for the treatment of organophosphate poisoning, such as intravenous atropine administration, are limited and not always available. Common antidotes used in OP exposure include atropine, which reduces the effect of the poisoning by blocking muscarinic acetylcholine receptors that in turn prevents the overstimulation of the receptors by excessive acetylcholine accumulation. Pralidoxime (2-PAM) another potential treatment works against organophosphate poisoning by re-cleaving the phosphorylation. In severe cases, benzodiazepines may be prescribed. Occasionally treatment with topical anticholinergic drugs induce mydriasis and have been shown to worsen visual acuity and visual function and is counterproductive to vision preservation particularly for war fighter. Direct identification of toxic compounds or nerve agents may not be readily available further complicating stabilization of patients and delivery of proper medical care (John et al., 2018; Steindl et al., 2021).

There is a need for the development of easy-to-administer non-toxic compounds that protect against organophosphate poisoning and preserve visual functioning. Ideally, such a treatment would be suitable for immediate use by first responders or active military personnel when at risk of toxic exposure to organophosphates (e.g., industrial disasters, anti-terrorism operations, or activities in active combat zones where OP agents may be used or present).

Tetrodotoxin (TTX) is a potent neurotoxin. Its name derives from Tetraodontiformes, an order that includes pufferfish, porcupinefish, ocean sunfish, and triggerfish. Although tetrodotoxin was discovered in these fish and found in several other animals, it is actually produced by certain infecting or symbiotic bacteria such as Pseudoalteromonas, Pseudomonas, and Vibrio.

Tetrodotoxin is a sodium channel blocker. It inhibits the firing of action potentials in neurons by binding to the voltage-gated sodium channels in nerve cell membranes and blocking the passage of sodium ions (responsible for the rising phase of an action potential) into the neuron. This prevents the nervous system from carrying messages and thus muscles from contracting in response to nervous stimulation.

Tetrodotoxin binds to what is known as site 1 of the fast voltage-gated sodium channel. Site 1 is located at the extracellular pore opening of the ion channel. The binding of any molecules to this site temporarily disables the function of the ion channel, thereby blocking the passage of sodium ions into the nerve cell.

TTX is extremely toxic. The oral median lethal dose (LD50) for mice as 334 μg per kg. For comparison, the oral LD₅₀ of potassium cyanide for mice is 8.5 mg per kg, demonstrating that even orally, TTX is more poisonous than cyanide.

The toxin can enter the body of a victim by ingestion, injection, or inhalation, or through abraded skin. Poisoning as a consequence of consumption of fish is extremely serious. The organs (e.g., liver) of the pufferfish can contain levels of tetrodotoxin sufficient to produce paralysis of the diaphragm and corresponding death due to respiratory failure.

Symptoms typically develop within 30 minutes of ingestion, but may be delayed by up to four hours; however, if the dose is fatal, symptoms are usually present within 17 minutes of ingestion. Paresthesia of the lips and tongue is followed by developing paresthesia in the extremities, hypersalivation, sweating, headache, weakness, lethargy, incoordination, tremor, paralysis, cyanosis, aphonia, dysphagia, and seizures. The gastrointestinal symptoms are often severe and include nausea, vomiting, diarrhea, and abdominal pain; death is usually secondary to respiratory failure. The victim, although completely paralyzed, may be conscious and in some cases completely lucid until shortly before death, which generally occurs within 4 to 6 hours. If the patient survives 24 hours, recovery without any residual effects will usually occur over a few days.

Therapy is supportive and based on symptoms, with aggressive early airway management. If ingested, treatment can consist of emptying the stomach, feeding the victim activated charcoal to bind the toxin, and taking standard life-support measures to keep the victim alive until the effect of the poison has worn off. Alpha adrenergic agonists are recommended in addition to intravenous fluids to combat hypotension.

### SUMMARY OF THE INVENTION

The present invention provides materials and methods for alleviating toxicity caused by organophosphates and other toxic materials, including tetrodotoxin (TTX) toxicity. More specifically, in certain embodiments the invention provides pharmaceutical compositions comprising a protein, and/or biologically-active fragments and variants thereof, and methods of using the same. In additional embodiments, the subject invention provides pharmaceutical compositions comprising bacteria grown as biofilm and/or components of bacterial biofilm, including, for example, cells and extracellular matrix.

In preferred embodiments, the present invention provides biologically-active proteins having the ability to prevent or reduce toxicity to organophosphates. In a specific embodiment, the present invention provides “Qi611S,” a protein having an amino acid sequence according to SEQ ID NO. 1. In certain embodiments, the present invention also provides “Qi611S Proteins,” which include Qi611 S, as well as biologically-active fragments and variants thereof.

In some embodiments, Qi611S Proteins can be produced by a cell, preferably a bacterial cell. Thus, in specific embodiments, the present invention provides methods for producing a Qi611S Protein, the methods comprise cultivating a cell having a nucleotide sequence that encodes all or a portion of SEQ ID NO. 1, or a variant or fragment thereof, under conditions favorable for expression of the protein. In preferred embodiments, the nucleotide sequence is Qi611s (SEQ ID NO. 2). Optionally, the protein can be purified from the culture.

In one embodiment, the methods utilize a microorganism, e.g., Lactobacillus fermentum Qi6, having the Qi611s nucleotide sequence (SEQ ID NO. 2). Qi611s encodes the amino acid sequence according to SEQ ID NO. 1 (Qi611S).

In another embodiment, the cell is a microorganism that has been recombinantly altered to possess the ability to express a Qi611 S Protein. In a specific embodiment, the microbe possesses all, or a portion, of the Qi611s gene. Thus, in certain embodiments, the present invention provides a recombinant cell possessing all or a portion of the DNA sequence according to SEQ ID NO. 2, and/or that is capable of expressing a protein having an amino acid sequence according to SEQ ID NO. 1, or a fragment or variant of SEQ ID NO. 1. In exemplary embodiments, the recombinant cell is E. coli BL21 or E. coli C43.

Such transformation of cells can be accomplished using techniques well known to those skilled in the microbiological arts. In one embodiment, the nucleotide sequence can be modified to optimize expression of a Qi611S Protein.

In preferred embodiments, the present invention provides compositions comprising a Qi611 S Protein and/or a cell comprising all or a portion of a DNA sequence according to SEQ ID NO. 2, and, optionally, a pharmaceutically acceptable carrier.

Additional preferred embodiments of the invention provide compositions, and methods of using the same, comprising a strain of Lactobacillus fermentum bacterium, and/or one or more bioactive extracts thereof, grown in biofilm phenotype. The subject invention also provides compositions comprising biofilm of L. fermentum bacterium, and/or bioactive extracts thereof, in a lyophilized, freeze dried, and/or lysate form.

Pharmaceutically acceptable carriers can comprise substances used for administrating the composition to a subject via a specific route, including, for example, oral administration, administration to the respiratory tract, administration to the eyes, injection (e.g., subcutaneous, intravenous, intramuscular, intraperitoneal, intrathecal, and/or subdermal), and/or topical administration (e.g., via dermal absorption). In certain embodiments, the composition can be formulated as a lotion, cream, emulsion, ointment, oil, gel, aerosol, mist, vapor, and/or combinations thereof.

In some embodiments, the protein can be extracted and, optionally, purified from a cell culture before being formulated into the pharmaceutical composition.

In some embodiments, the composition comprises a cell capable of producing a Qi611S Protein. In one embodiment, the cell is in a lyophilized, freeze dried, and/or lysate form. In one embodiment, the composition comprises a cell culture in a biofilm state.

Thus, in certain embodiments, the present compositions can be used for treating and/or preventing organophosphate toxicity or TTS toxicity in a subject.

The subject invention further provides assays for assessing the toxicity induced by organophosphates as well as the efficacy of agents in preventing and/or reducing that toxicity.

### BRIEF DESCRIPTION OF THE FIGURES

FIG. 1 shows a map of the pET-15b vector utilized according to embodiments of the present invention.

FIG. 2 shows the pET-15b cloning/expression region of the coding strand transcribed by T7 RNA polymerase.

FIG. 3 shows a silver-stained SDS-PAGE gel used to establish the presence of Qi611 S synthesized by a recombinant E. coli strain. The SDS-PAGE gel was loaded with samples of released protein from the recombinant E. coli BL21, which are identified by the bold arrow at the right of the gel. The E. coli strain was transformed with the Qi611s gene via the pET-15b expression vector.

FIG. 4 shows a hydrophilicity plot of Qi611S.

FIG. 5 shows that saline, DMSO, or Qi611S alone do not significantly affect pupil sizes of ex vivo rabbit eyes.

FIG. 6 shows that a saline solution of Qi611S compound does not cause pupillary miosis within two hours posttreatment.

FIG. 7 shows that paraoxon is capable of decreasing pupil size in a rabbit ex vivo model, reproducing miosis seen typically as one of the hallmarks of organophosphate poisoning. Pictures were taken 10 min after corneal surface exposure to pOX, with the nylon rings containing the treatment applied to the corneal-limbal area.

FIG. 8 shows compound Qi611S instigate protection against paraoxon-induced miosis in juvenile rabbit ex vivo eye model FIG. 9 shows that prophylactic treatment with Qi611S protects AChE activity from paraoxon-induced inhibition.

FIG. 10 shows that protection of AChE activity by Qi611S is rapid as demonstrated with 10- and 30-min prophylactic treatment before paraoxon exposure.

FIG. 11 shows that upregulation of PPAR-γ correlates with the protection of AChE activity in ex vivo rabbit eyes treated with Qi611 S. Paraoxon (P) at 1 mM and 10 mM concentrations were used in this experiment.

FIG. 12 shows prophylactic treatment with Qi611S before paraoxon exposure decreases tissue damage as demonstrated with LDH release assay.

### BRIEF DESCRIPTION OF THE SEQUENCES

SEQ ID NO: 1 is the amino acid sequence of the protein designated as “Qi611S.”
SEQ ID NO: 2 is Qi611s, a nucleotide sequence encoding the amino acid sequence of SEQ ID NO: 1.
SEQ ID NO: 3 is a forward primer, “Lacto F,” useful according to the subject invention.
SEQ ID NO: 4 is a reverse primer, “Lacto R,” useful according to the subject invention.
SEQ ID NO: 5 is the nucleotide sequence for the cloning/expression region of the pET-15b vector.
SEQ ID NO: 6 is an amino acid sequence for a His-tagged recombinant protein encoded by the cloning/expression region of the pET-15b vector.

### DETAILED DESCRIPTION OF THE INVENTION

The present invention provides materials and methods for alleviating organophosphate toxicity. More specifically, the invention provides pharmaceutical compositions comprising a protein, and/or biologically-active fragments and variants thereof, and methods of using the same.

The present invention provides a protein (“Qi611S”) having an amino acid sequence according to SEQ ID NO: 1, biologically-active fragments and variants thereof, as well as therapeutic compositions and methods that utilize the novel protein and/or its biologically-active fragments and variants to reduce organophosphate toxicity.

Further embodiments provide cells that are capable of expressing a Qi611 S protein. In certain embodiments, the cells are recombinantly altered to possess a nucleotide sequence that encodes all or a portion of SEQ ID NO: 1. In a specific embodiment, the nucleotide sequence has SEQ ID NO: 2.

In some embodiments, the bacterial strain is Lactobacillus fermentum Qi6, also referred to herein as Lf Qi6. In one embodiment, the subject invention provides an isolated or a biologically pure culture of Lf Qi6. In another embodiment, the subject invention provides a culture of Lf Qi6, grown as a biofilm. Specifically taught herein are methods for inducing and identifying the biofilm phenotype. Further provided herein are methods of utilizing the biofilm phenotype, as well as extracts of the biofilm phenotype, and lysates thereof. In specific embodiments, the pharmaceutical compositions comprise bioactive extracts of Lf Qi6 biofilm.

A culture of the L. fermentum microbe has been deposited with the American Type Culture Collection (ATCC), 10801 University Blvd., Manassas, Va. 20110-2209 USA. The deposit has been assigned accession number ATCC No. PTA-122195 by the repository and was deposited on Jun. 10, 2015.

In certain embodiments, the compositions comprise a strain of bacteria, preferably Lactobacillus fermentum bacteria, grown as biofilm. The subject invention also provides compositions comprising extracts or extracellular matrix of biofilm, in a lyophilized, freeze dried, and/or lysate form.

The subject invention further provides an ex vivo ocular model that, in preferred embodiments, uses the simultaneous quantitative evaluation of two pathognomonic endpoints in organophosphate (OP) poisoning: pupillary response (miosis) and acetylcholinesterase inhibition. Using this model and paraoxon, five characteristics of Qi611S in the setting of ocular OP challenge were demonstrated: 1) physiologic protection due to reduced maximal miosis recorded after paraoxon challenge, 2) protection of AChE enzymatic activity, 3) rapid onset of physiologic and enzymatic protection (10 minutes pretreatment of ex vivo eyes with Qi611S), 4) no toxicity of Qi611S to acetylcholinesterase or attenuation of pupillary response, and 5) the presence of Qi611S in the intraocular fluid within 30 minutes of application.

Other advantages of the model for OP toxicity extend to the feasibility of the use of whole ex vivo eyes and fast onset of measurable outcomes as paraoxon-induced miosis was visible within minutes from application of the test OP compound. Additionally, the amount of anterior chamber fluid recovered per each eye is sufficient to perform several different tests with eye tissues (e.g., corneal buttons after punch biopsy) being available for microscopy or immunohistochemistry. Because the Qi611S Proteins employ multi-functional physiologic host-protective mechanisms mitigating organophosphate toxicity, these compounds can be used for broad-spectrum protection against multiple organophosphate agents not only for the eye but also provide tissue barrier protection for, for example, the skin and respiratory tract.

### Selected Dentitions

“Qi611 S” refers to a protein having the amino acid sequence of SEQ ID NO: 1. Reference to a “Qi611 S Protein,” in the singular or plural, refers to Qi611S, as well as biologically-active fragments and variants of 611 S.

As used herein, “gene” refers to a segment of DNA, or a nucleotide sequence, capable of expressing a polypeptide and/or amino acid chain. In certain embodiments, the gene includes regions, such as promoter regions, preceding and/or following a coding region.

As used herein, an “isolated” or “purified” compound is substantially free of other compounds, such as cellular material, with which it is associated in nature. A purified or isolated polynucleotide (ribonucleic acid (RNA) or deoxyribonucleic acid (DNA)) is free of sequences that flank it in its naturally-occurring state. A purified or isolated polypeptide is free of, for example, other cellular material with which it would be associated in nature. “Isolated” in the context of a microbial strain means that the strain is removed from the environment in which it exists in nature. Thus, the isolated strain may exist as, for example, a biologically pure culture, or as spores (or other forms of the strain) in association with a carrier.

As used here in, a “biologically pure culture” is one that has been isolated from other biologically active materials, including any materials with which it may have been associated in nature. In a preferred embodiment, the culture has been isolated from all other living cells. In further preferred embodiments, the biologically pure culture has advantageous characteristics compared to a culture of the same microbial species that may exist in nature. The advantageous characteristics can be, for example, enhanced production of one or more desirable growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is preferably one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. Examples of metabolites include, but are not limited to, biosurfactants, biopolymers, enzymes, acids, solvents, alcohols, proteins, vitamins, minerals, microelements, and amino acids.

As used herein the term “extract” refers to a composition obtained by processing a microbial culture. The processing may involve, for example, physical and/or chemical treatment. The physical and/or chemical treatment may comprise, for example, filtering, centrifugation, sonication, pressure treatment, radiation treatment, lysing, treatment with solvents or other chemicals, and combinations of these treatments. The extract can be in the form of, for example, a supernatant such as that produced via centrifugation. The extract can also include cell mass obtained through centrifugation. The cells may be intact or not intact, viable or not viable. The extract may comprise cell membrane components and/or intracellular components. In certain embodiments, the extract is at least 80, 85, 90, or 95%, by weight, cell mass. In certain embodiments, at least 95% of the intact cells are non-viable. In certain embodiments, less than 10% of the cell mass in the extract is intact cells.

“Planktonic” refers to a phenotype typical to microorganisms (bacteria, fungi, and/or protozoa, with associated bacteriophages and other viruses) that float freely in a liquid medium. A “biofilm,” on the other hand, is an accumulation of microorganisms embedded in an extracellular polymeric matrix (EPS) and adherent to solid biological or non-biotic surfaces. Lf Qi6 can form biofilm in addition to possessing the typical planktonic phenotype.

Methods for growing biofilm are known in the art and are described in, for example, WO 2012/118535, which is incorporated herein, in its entirety, by reference, including the publications cited in that reference, such as those cited at pages 26-31.

As used herein, “modulate” means to cause an alteration (e.g., increase or decrease).

As used herein, a “pharmaceutical,” “health-promoting compound,” or “health-promoting substance” refers to a compound that is useful as a medicinal and/or therapeutic drug.

As used herein, a “polypeptide,” “peptide” or a “protein” refers to a polymer of amino acid residues.

As used herein, the term “subject” refers to an animal. The animal may be, for example, a human, pig, horse, goat, cat, mouse, rat, dog, ape, fish, chimpanzee, orangutan, guinea pig, hamster, cow, sheep, bird (including chicken), as well as any other vertebrate or invertebrate.

The preferred subject, in the context of this invention, is a human of any age and/or gender. In some embodiments, the subject is suffering from a health condition, disease, or disorder caused by organophosphate toxicity, while, in some embodiments, the subject is in a state of good health (e.g., free from injury or illness) but desires protection against organophosphate toxicity.

As used herein, the terms “treating” and “treatment” refer to eradicating, reducing, ameliorating, or reversing a sign or symptom of a health condition, disease, or disorder to any extent and includes, but does not require, a complete cure of the condition, disease, or disorder. Treating can be curing, improving, or partially ameliorating a disorder. Treatment can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required to, be absolute or complete, meaning the sign or symptom may still develop at a later time. Prevention can include reducing the severity of the onset of such a condition, disease, or disorder and/or inhibiting the progression of the condition, disease, or disorder to a more severe condition, disease, or disorder.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “recombinant” cells are modified by the introduction of a heterologous nucleic acid molecule, or the alteration of a native nucleic acid molecule. Thus, for example, recombinant cells can express genes that are not found within the native (non-recombinant) form of the cell or express native genes that are otherwise abnormally expressed, under-expressed or not expressed at all.

As used herein, “reduce” refers to a negative alteration, and the term “increase” refers to a positive alteration, each of at least 1%, 5%, 10%, 25%, 50%, 75%, or 100%.

As used herein, “reference” refers to a standard or control condition.

The transitional term “comprising,” which is synonymous with “including,” or “containing,” is inclusive or open-ended and does not exclude additional elements or method steps not recited. By contrast, the transitional phrase “consisting of” excludes any element, step, or ingredient not specified in the claim. The transitional phrase, “consisting essentially of,” limits the scope of a claim to the specified materials or steps “and those that do not materially affect the basic and novel characteristic(s)” of the claimed invention, e.g., the ability to preclude bacterial growth. Use of the term “comprising” contemplates embodiments “consisting” and “consisting essentially” of the recited component(s).

Unless specifically stated or is obvious from context, as used herein, the term “or” is understood to be inclusive. Unless specifically stated or is obvious from context, as used herein, the terms “a,” “an,” and “the” are understood to be singular or plural.

Unless specifically stated or is obvious from context, as used herein, the term “about” is understood as within a range of normal tolerance in the art, for example, within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof and from the claims. All references cited herein are hereby incorporated by reference in their entirety.

### Organophosphates

Organophosphates are a class of organophosphorus compounds with the general structure O═P(OR)3, a central phosphate molecule with alkyl or aromatic substituents.

Organophosphate pesticides, like some nerve agents, inhibit acetylcholinesterase, which is broadly essential for normal function in insects, but also in humans and many other animals. Organophosphates affect this enzyme in varied ways, a principal one being through irreversible covalent inhibition, and thus create the potential for poisoning in varying degrees. The brain sends out neurotransmitters to the nerve endings in the body; organophosphates disrupt this process from occurring. Acetylcholinesterase break down the acetylcholine neurotransmitter, which sends out signals to other nerve endings in the body.

Commonly used organophosphates have included:
parathion
malathion
methyl parathion
chlorpyrifos
diazinon
dichlorvos
phosmet
fenitrothion
tetrachlorvinphos
azamethiphos
azinphos-methyl
terbufos

In specific embodiments, organophosphates have the following structural features:
A terminal oxygen connected to phosphorus by a double bond, i.e., a phosphoryl group;
Two lipophilic groups bonded to the phosphorus; and
A leaving group bonded to the phosphorus, often a halide.

Many organophosphates are potent nerve agents, functioning by inhibiting the action of acetylcholinesterase (AChE) in nerve cells. The first such nerve agents that were made were called G series and included Sarin, Tabun, and Soman. Later work produced VX nerve agents, which were a magnitude more potent than the G series. When exposed to VX or G series toxins the body's signaling mechanisms between the nervous and muscular systems become disrupted. This causes neuromuscular blockage, flaccid paralysis and death by asphyxiation. Novichok is another organophosphate nerve agent. Organophosphate nerve agents are described in more detail by Mukherjee et al., incorporated herein, in its entirety by reference.

### Qi611S Proteins and Polynucleotide Sequences Encoding 611S Proteins

In preferred embodiments, the present invention provides a protein, as well as fragments and variants thereof, useful for ameliorating toxicity to organophosphates. The present invention further provides nucleotide sequences that encode the protein, as well as fragments and variants thereof.

In certain specific embodiments, a protein of the present invention, referred to as “Qi611 S,” has a molecular weight of about 8.0 kDA. “Qi6l 1S proteins”, which include Qi6l S and biologically-active fragments and variants thereof, can be characterized according to several parameters, including biological activities, such as, for example: reduction in organophosphate toxicity; antimicrobial activity; inhibiting pathogenic biofilm growth and adhesion; promoting pathogenic biofilm detachment; promoting commensal biofilm growth; modulating metabolism; enhancing skin barrier functions and innate immune functions; inhibiting cancer cell proliferation; and/or inducing expression and/or activity of various receptors and/or kinases. In specific embodiments, Qi611S proteins have anti-cancer, antimicrobial (e.g., antibacterial, antifungal and/or antiviral), anti-inflammatory, metabolism modulating and/or skin immunomodulatory activity.

In certain embodiments, Qi611S Proteins can, directly or indirectly, induce expression of, and/or act as an agonist toward, one or more molecules selected from, for example, peroxisome proliferator-activated receptors (PPARs) (e.g., PPARα, PPARβ/δ, and/or PPARγ); extracellular signal-regulated kinases (ERK 1/2); and glucocorticoid receptors (GR). Whether the Qi611S Proteins act as inducers or agonists can depend upon, for example, the biological or metabolic systems and/or the type(s) of cells (e.g., bacteria, cancer or immune cells) involved.

A Qi611 S Protein can further be defined by its amino acid sequence. In a specific embodiment (Qi611 S), the protein has the 74 amino acid sequence shown as SEQ ID NO: 1.

In certain embodiments, the proteins provided herein can also be identified based on immunoreactivity with certain antibodies, as well as other methods described below.

In certain embodiments, Qi611S Proteins are produced by the Lactobacillus fermentum Qi6 bacterial strain when laboratory growth conditions are used to force the growth into a biofilm phenotype. In preferred embodiment, this bacterial strain possesses the Qi611S DNA sequence (SEQ ID NO: 2), which is capable, under biofilm phenotype conditions, of expressing a protein having SEQ ID NO: 1.

In further embodiments, a polynucleotide encoding a Qi611S Protein can be defined by, for example, the ability to hybridize with, or be amplified by, certain exemplified probes and primers (e.g., SEQ ID NOs: 3-4).

Lactobacillus fermentum is a Gram-positive rod. Lactobacillus fermentum Qi6 (Lf Qi6) can be grown in MRS media at 37° C.

A culture of the L. fermentum Qi6 microbe has been deposited with the American Type Culture Collection (ATCC), 10801 University Blvd., Manassas, Va. 20110-2209 USA. The deposit has been assigned accession number ATCC No. PTA-122195 by the repository and was deposited on Jun. 10, 2015.

The subject culture has been deposited under conditions that assure that access to the culture will be available during the pendency of this patent application to one determined by the Commissioner of Patents and Trademarks to be entitled thereto under 37 CFR 1.14 and 35 U.S.C 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In some embodiments, Lf Qi6 can be grown in a biofilm phenotype. As used herein, a “biofilm” is a complex aggregate of microorganisms, such as bacteria, wherein the cells adhere to each other using a matrix usually composed of, but not limited to, polysaccharide material. The cells in biofilms have physiologically distinct properties compared to planktonic cells of the same organism, which are single cells that can float or move about in liquid or gaseous mediums, or reside on or in solid or semi-solid surfaces.

In certain embodiments, the present invention provides isolated polynucleotide sequences, or genes, that encode the therapeutic and/or cosmetically-useful Qi611 S Proteins. Furthermore, in some embodiments, the present invention provides methods for using the polynucleotide sequences to produce recombinant hosts that express a Qi611S Protein.

In certain embodiments, the polynucleotide sequence is Qi611S, which is 222 base pairs and encodes Qi611 S; however, in certain embodiments, different DNA sequences can encode the amino acid sequences disclosed herein because of, for example, the redundancy of the genetic code. It is well within the skill of a skilled artisan to create these alternative DNA sequences encoding the Qi611 S Proteins.

As used herein, “variants” of a protein refer to sequences that have one or more amino acid substitutions, deletions, additions, or insertions. In preferred embodiments, these substitutions, deletions, additions or insertions do not materially adversely affect the therapeutic activity of Qi611 S. Variants that retain one or more biological activities of Qi611S are within the scope of the present invention. Preferably the one or more biological activities are selected from reduction in organophosphate toxicity; antimicrobial activity; inhibiting pathogenic biofilm growth and adhesion; promoting pathogenic biofilm detachment; promoting commensal biofilm growth; modulating metabolism; enhancing skin barrier functions and innate immune functions; inhibiting cancer cell proliferation; and/or inducing expression and/or activity of various receptors and/or kinases.

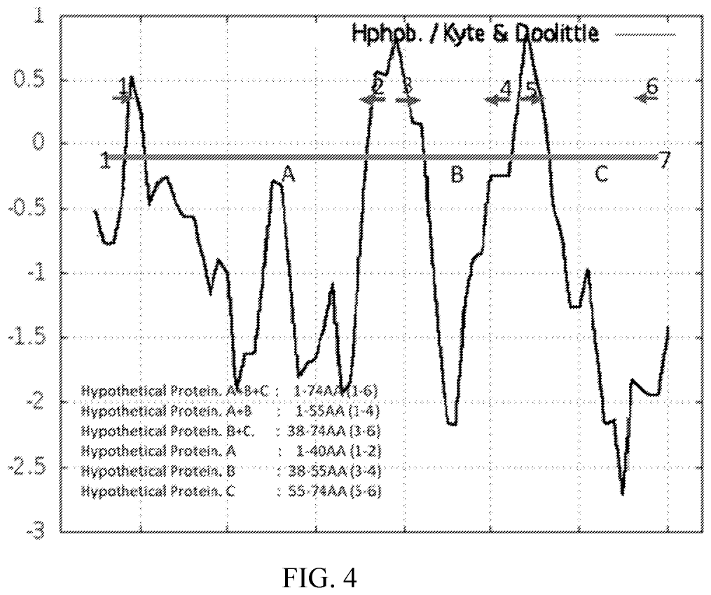

“Fragments” of Qi611S and its variants are also within the scope of Qi611 S Proteins, so long as the fragment retains one or more biological properties of Qi611 S. Preferably the one or more biological activities are selected from reduction in organophosphate toxicity; antimicrobial activity; inhibiting pathogenic biofilm growth and adhesion; promoting pathogenic biofilm detachment; promoting commensal biofilm growth; modulating metabolism; enhancing skin barrier functions and innate immune functions; inhibiting cancer cell proliferation; and/or inducing expression and/or activity of various receptors and/or kinases. Preferably, the fragment is at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the full length Qi611S. The fragment may comprise, for example, one or more hydrophilic domains of Qi611S or variant. These domains may be directly connected with intervening amino acids removed. Hydrophilic domains can be readily identified using standard procedures known in the art and as exemplified in FIG. 4.

The subject invention further contemplates fusion constructs where a Qi611S Protein is attached, directly or indirectly (e.g., via a linker), to another moiety that may be, for example, a targeting moiety (e.g., ligand, antibody, or aptamer), a toxin, a carrier, a label, or an activity enhancer.

The subject invention further contemplates antibodies (e.g., polyclonal, monoclonal, chimeric, and humanized) to the Qi611 S Proteins. These antibodies can be readily prepared by a person of ordinary skill in the art having possession of the teachings provided herein. These antibodies can be used for, for example, therapies, diagnostics, and protein purification.

In certain embodiments, a polynucleotide encoding a Qi611S Protein can be isolated, amplified and ligated into a vector. A “vector,” “plasmid,” or “plasmid vector” is a DNA molecule used to transfer DNA to a cell, often from one cell to another (a host cell). The vector can be replicated in the host cell; or, the vector can be a means to incorporate DNA into (or remove DNA from) a cell. A variety of means can be used to introduce a vector into a host cell. Some cells can uptake a vector without any action by one skilled in the art other than placing the vector in the cell culture. Others require chemical modification. Regardless of the means with which a cell can uptake a vector, once a host cell has the ability to do so, it is now a “competent” cell.

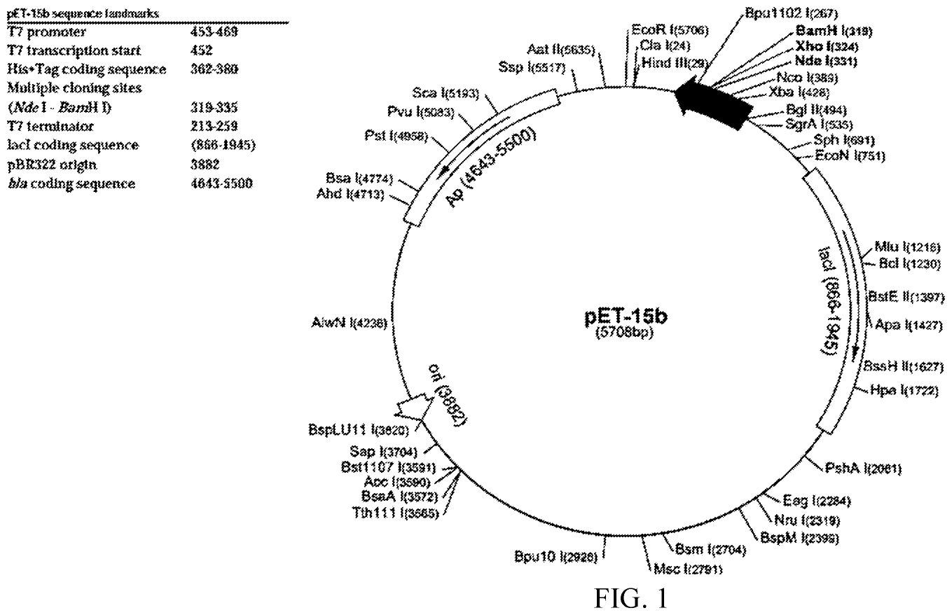

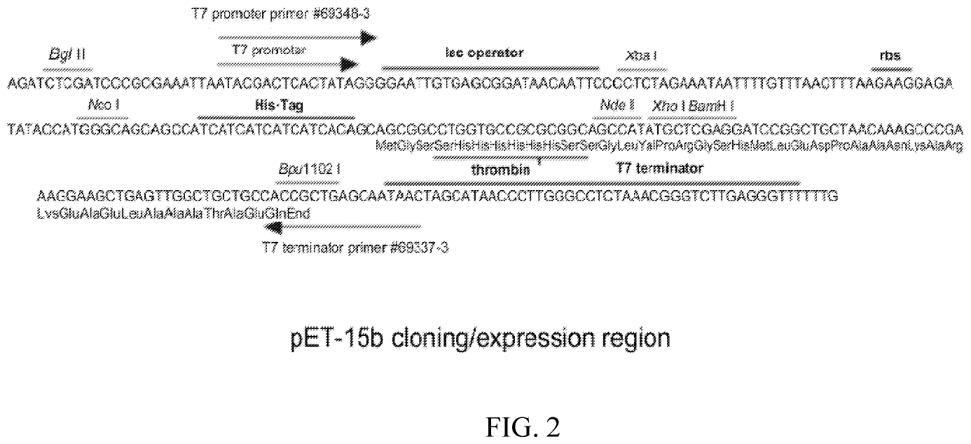

One example of a commercially available vector is pET-15b, in which, using restriction enzyme digestion, one skilled in the art can create a vector carrying Qi611S, or other polynucleotide encoding a Qi611S Protein. The pET-15b vector carries an N-terminal His·Tag® sequence followed by a thrombin site and three cloning sites (SEQ ID NO. 6). Unique sites are shown on the circle map depicted in FIG. 1. The cloning/expression region of the coding strand transcribed by T7 RNA polymerase is shown in FIG. 2 (SEQ ID NO. 5).

In certain embodiments, the present invention pertains to the genetic transformation of host cells so as to provide these cells with the ability to produce a Qi611 S protein. For example, a vector with Qi611S (or other polynucleotide encoding a Qi611 S Protein) can be transformed into a host cell (e.g., a microorganism, a plant, a fungal, and/or an animal cell) allowing for the use of recombinant cells for the production of the Qi611S Protein.

In preferred embodiments, the host cell is a strain of Escherichia coli, e.g., E. coli BL21 or E. coli C43. Alternatively, the ability to transform cells, other than E. coli, into competent cells is well understood in the art, this includes cells chosen based on, e.g., their transformation ability, ability and efficiency for heterologous protein expression, stability of the protein in the host, presence of auxiliary genetic capabilities, lack of mammalian toxicity, ease of killing and fixing without damage to the protein, ease of cultivation and/or formulation, ease of handling, economics, storage stability and the like.

Bacterial hosts of particular interest include, for example, Escherichia, Lactobacillus, and Bacillus.

Eukaryotic hosts include, for example, Phycomycetes and Ascomycetes, which includes yeast, such as Saccharomyces and Schizosaccharomyces; and Basidiomycetes yeast, such as Rhodotorula, Aureobasidium, Sporobolomyces, and the like.

The pET-15b vector, for example, encodes a polyhistidine-tag and a thrombin cleavage site. The synthesis can be assessed through methods such as a polyhistidine-tag encoded alongside the protein to enable affinity chromatography, also referred to as affinity purification. The tag can be left attached to the Qi611S Protein synthesized by the host, or, preferably, it can be cleaved off the protein by means of the thrombin cleavage site before the recombinant protein is used.

Also within the scope of the subject instant invention are vectors or expression cassettes containing genetic constructs as set forth herein or polynucleotides encoding the polypeptides, set forth supra, operably linked to regulatory elements. The vectors and expression cassettes may further comprise selectable markers. The vectors and expression cassettes may contain additional transcriptional control sequences as well, such as, for example, strong promoters to enhance expression of cloned genes.

The expression cassette will typically include in the 5′-3′ direction of transcription, a transcriptional and translational initiation region, a coding sequence of the present invention, and a transcriptional and translational termination regions. The transcriptional initiation region, the promoter, may be native or analogous, or foreign or heterologous, to the host cell. By “foreign” is intended that the transcriptional initiation region is not found in the organism into which the transcriptional initiation region is introduced.

The subject invention also provides detection probes (e.g., fragments of the disclosed polynucleotide sequences) for hybridization with a target sequence or the amplicon generated from the target sequence. Such a detection probe will comprise a contiguous/consecutive span of at least 8, 9, 10, 11, 12, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 nucleotides of SEQ ID NO: 2.

The present invention also provides methods of producing a Qi611S Protein by cultivating a host cell transformed with a polynucleotide of the present invention (e.g., SEQ ID NO: 2) under conditions that allow for the expression of the polypeptide and, optionally, recovering the expressed polypeptide.

In certain embodiments, the host cell is transformed to express the polypeptide in enhanced amounts. For example, the DNA vector can include a strong transcriptional promoter sequence closely preceding the gene to be cloned. In certain embodiments, the strong promoter is a trp operon, a lac operon, a T7 promoter, and/or a pL promoter.

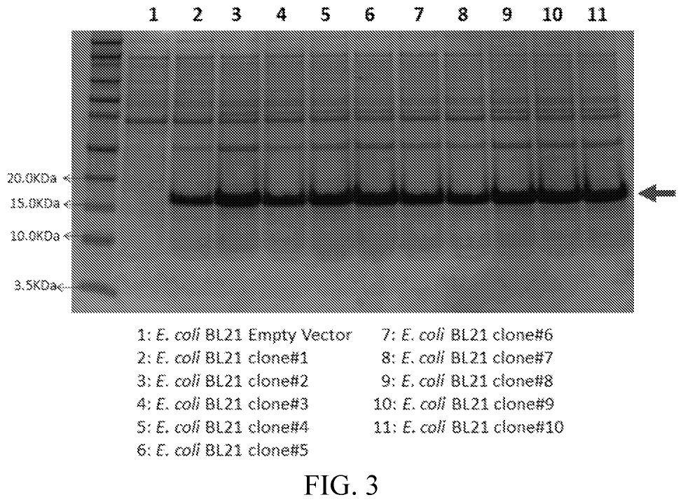

FIG. 3 shows a silver-stained SDS-PAGE gel used to establish the presence of Qi611S synthesized by a recombinant E. coli strain. The SDS-PAGE gel was loaded with samples of released protein from the recombinant E. coli BL21. The E. coli strain was transformed with the Qi611S gene via the pET-15b expression vector.

In certain embodiments, a Qi611S Protein can be purified from the culture in which it is synthesized through the use of tags such as polyhistidine tags and Glutathione-S-Transferase. Other means of protein purification can be used in conjunction with affinity chromatography or without affinity chromatography. Some alternative methods include centrifugation, filtration, sonication, and fractionation. The protein can be precipitated out of culture through the addition of ammonium sulfate. Also, various other chromatographic methods can be used, such as ion exchange chromatography, hydrophobic interaction chromatography, reversed phase chromatography, or immobilized metal affinity chromatography. These protein purification methods can be used in isolation or in conjunction with each other. The method or combination of methods has various advantages and disadvantages that are understood by those skilled in the art.

It will be recognized by those skilled in the art that DNA sequences of the subject invention may vary due to the degeneracy of the genetic code and codon usage. All DNA sequences that encode a Qi611S Protein are contemplated. Thus, all polynucleotide sequences that encode a Qi611S Protein are included in this invention, including DNA (optionally including an ATG preceding the coding region) that encodes SEQ ID NO: 1. The subject invention also includes polynucleotides having codons that are optimized for expression in a host cell, including any of the specific types of cells referred to herein. Various techniques for creating optimized sequences are known in the art.

Additionally, it will be recognized by those skilled in the art that allelic variations may occur in the DNA sequences that will not significantly change activity of the amino acid sequences of the peptides that the DNA sequences encode. All such variant DNA sequences are included within the scope of this invention.

The skilled artisan will understand that the exemplified sequences can be used to identify, produce, and use additional nucleotide sequences that encode Qi611S Proteins. Variant DNA sequences having at least 90%, or at least 95% identity to a recited DNA sequence and encoding a Qi611S Protein are included in the subject invention. Other numeric ranges for variant polynucleotides and amino acid sequences are provided below (e.g., 50-99%). Following the teachings herein and using knowledge and techniques well known in the art, the skilled worker will be able to make a large number of operative embodiments having variant DNA sequences without the use of undue experimentation. Specifically contemplated are homologs from other strains or species.

The fragments and the mutational, insertional, and deletional variants of the polynucleotide and amino acid sequences of the invention can be used in the same manner as the exemplified sequences so long as the fragments and variants have substantial sequence similarity with the original sequence. As used herein, substantial sequence similarity refers to the extent of nucleotide or amino acid sequence similarity that is sufficient to enable the variant or fragment sequence to function in the capacity as the original sequence. Preferably, this similarity is greater than 50%; more preferably, this similarity is greater than 75%; and most preferably, this similarity is greater than 90%. The degree of similarity needed for the variant to function in its intended capacity will depend upon the intended use of the sequence. It is well within the skill of a person trained in this art to make mutational, insertional, and deletional mutations that are designed to improve the function of the sequence or otherwise provide a methodological advantage. The identity and/or similarity can also be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein.

The amino acid identity/similarity and/or homology will typically be highest in critical regions of the protein that account for biological activity and/or are involved in the determination of three-dimensional configuration that ultimately is responsible for the biological activity. In this regard, certain amino acid substitutions are acceptable and can be expected if these substitutions are in regions that are not critical to activity or are conservative amino acid substitutions that do not affect the three-dimensional configuration of the molecule. For example, amino acids may be placed in the following classes: non-polar, uncharged polar, basic, and acidic. Conservative substitutions, whereby an amino acid of one class is replaced with another amino acid of the same type, fall within the scope of the subject invention so long as the substitution does not materially alter the biological activity of the compound. The following (Table 1) is a list of examples of amino acids belonging to each class.

TABLE 1
Classification of amino acids based on physical properties.

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the biological activity of the protein.

### Therapeutic Uses for Qi611S Proteins, Cell Cultures, Cell Components and Extracts

The Qi611S Proteins, cell cultures, cell components and extracts of the present invention can be useful for therapeutic and/or prophylactic applications.

“Therapeutic,” as used herein, means useful for treating and/or preventing a disease, condition or disorder.

In preferred embodiments, the invention provides pharmaceutical compositions comprising a Qi611 S Protein (e.g., SEQ ID NO: 1) and/or a cell capable of producing a Qi611 S Protein (as well as components of such cells), as well as methods of using the same. In certain embodiments, the compositions can optionally comprise a pharmaceutically-acceptable carrier.

In further embodiments, the invention provides methods of providing a therapeutic benefit to a subject in need therein, wherein a composition according to the present invention is administered to the subject. In some embodiments, a therapeutically-effective dose of the composition may be administered to the subject, e.g., one, two, three, or more times daily, for as long as needed, or until the desired benefit is achieved.

As used herein, a “therapeutically-effective” amount or dose is an amount or dose of a compound or composition that, when administered to a subject, is capable of treating or improving a condition, disease, or disorder in a subject or that is capable of providing enhancement in health or function to an organ, tissue, or body system. Preferably, the condition is organophosphate toxicity.

The actual amount will vary depending on a number of factors including, but not limited to, the particular condition, disease, or disorder being treated or improved; the severity of the condition; the particular organ, tissue, or body system of which enhancement in health or function is desired; the weight, height, age, and health status of the patient; and the route of administration. Prescription of treatment, e.g., decisions on dosage etc., is within the purview of general practitioners and other medical doctors, and typically takes into account the disorder to be treated, the condition of the individual patient, the site of delivery, the method of administration and other factors known to practitioners.

In certain embodiments, the therapeutic benefit is treatment and/or prevention of organophosphate toxicity.

In some embodiments, the compositions and methods utilize a biologically pure culture of a cell capable of producing a Qi611 S Protein. The cell can be a microorganism that possesses SEQ ID NO: 2, such as, for example, L. fermentum Qi6; and/or the cell can be a recombinant cell engineered to have a polynucleotide sequence that encodes a Qi611 S Protein, e.g., SEQ ID NO: 2. In an exemplary embodiment, the recombinant cell is E. coli BL21 or E. coli C43.

In certain embodiments, the cell is a bacterial strain capable of growing in both planktonic and biofilm phenotypes. In one embodiment, the cell is in a lyophilized, freeze dried, and/or lysate form.

In one embodiment, the protein can be extracted and, optionally, purified from a cell culture before use in the present compositions and methods. As used herein, the term “extracting” refers to processing a cell culture to obtain one or more desired compounds. The processing may involve, for example, physical and/or chemical treatment including, for example, filtering, centrifugation, sonication, pressure treatment, radiation treatment, lysing, treatment with solvents or other chemicals, and combinations of these treatments. The resulting extract can be in the form of, for example, a supernatant such as that produced via centrifugation. The extract can also include cell mass obtained through centrifugation. The cells may be intact or not intact, viable or not viable. The extract may comprise cell membrane components and/or intracellular components. In certain embodiments, the extract is at least 80, 85, 90, or 95%, by weight, cell mass. In certain other embodiments, the extract is at least 80, 85, 90 or 95%, by weight, protein.

The compositions provided herein may contain a single (unit) dose of cells, or lysate, or protein extracted therefrom. In some embodiments, a composition according to the present invention may comprise a dose of at least about 0.01% to about 100%, by weight, of a Qi611S protein, or at least about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0%, about 55%, about 60%, about 65%, about 70%, about 75%, about 80%, about 85%, about 90%, about 95%, or about 99% by weight of a Qi611 S protein.

In some embodiments, the compositions may comprise a dose of at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to about 5%, by weight of the Qi611S protein.

Suitable doses of bacteria (intact, lysed or extracted) may be in the range 10⁴ to 10¹² CFU, e.g., one of 10⁴ to 10¹⁰, 10⁵ to 10⁸, 10⁶ to 10¹², 10⁶ to 10¹⁰, or 10⁶ to 10⁸ CFU. For the purpose of the present invention the abbreviation CFU shall designate a “colony forming unit” that is defined as the number of bacterial cells as revealed by microbiological counts on agar plates.

### Anti-Inflammatory Activity and Immune Modulation

In some embodiments, a Qi611S Protein and/or cells comprising a nucleotide sequence encoding a Qi611S Protein can modulate the expression of skin innate immune peptides and/or cytokines involved in inflammation (e.g., PPARγ, interleukin-10 (IL-10), tumor necrosis factor alpha (TNFα), PERK, toll-like receptors (TLR), and/or filaggrin). Thus, in certain embodiments, the present compositions and methods can be used to enhance skin, and other, innate immune functions. Thus, in one embodiment, the compositions and methods can enhance skin barrier function by upregulating the expression of skin barrier proteins.

Advantageously, the compositions of the subject invention can be administered systemically or locally and can be used to reduce the dose of glucocorticoids required which mitigates side effects including immunosuppression.

### Dermatologic Uses

Human skin comprises two compartments, the deep compartment (the dermis) and the surface compartment (the epidermis). The skin constitutes a barrier against external attacks, particularly chemical, mechanical, or infectious attacks, as well as a number of defensive reactions against environmental factors such as, for example, climate, ultraviolet rays, and tobacco, and/or xenobiotic factors, such as, for example, microorganisms. This property is referred to as the skin barrier function and is mainly provided by the outermost layer of the epidermis, namely the stratum corneum. Detrimental changes in the barrier can be reflected by, for example, cutaneous discomfort, sensory phenomena and/or cutaneous dryness.

In some embodiments, repair or regeneration of the barrier includes repair or regeneration of a mucous membrane. Mucous membranes include mucosa of the mouth (including mucosa of the cheek, the soft palate, the tongue, including the under surface of the tongue and the floor of the mouth), the nose, the throat (including mucosa of the pharynx, the larynx, the trachea and the esophagus), the bronchi, the lungs, the eye, the ear, the gastrointestinal tract, the vagina, the penis, the urethra, the bladder, and the anus.

The active agent, e.g., a Qi611 S Protein can be present at 0.01% to 2%, or any range therebetween, including, for example, 0.1 to 0.5%.

### Ophthalmologic Uses

There is a need in the ophthalmic field for a non-GC GR-activator that does not damage ocular tissue, increase ophthalmic pressures, or predispose to glaucoma or cataract formation. These are all known complications of GC use. A non-GC GR-activator in combination with PPAR activation would be of particular use in the topical and intraocular treatment of ocular inflammation and ocular disease. PPAR signaling is an important activation pathway for the genesis and maintenance of several ocular tissue types including meibomian glands, conjunctival goblets cells, corneal limbal stem cells, trabecular meshwork, uveal tissue and choroidal membranes. Furthermore, the angiotensin receptor can be activated by PPAR gamma ligands, of which this unique peptide may act.

### Administration to a Subject

The present invention provides methods of providing a therapeutic benefit to a subject in need thereof, where a therapeutically-effective amount of a composition comprising a Qi611S Protein and/or a cell capable of producing a Qi611S Protein, is administered to a subject in need thereof. In some embodiments, the composition can comprise, and/or be administered concurrently with, a pharmaceutically-acceptable carrier.

In one embodiment, the subject has already been exposed to an organophosphate. Such exposure may be known by, for example, observance of the exposure, detection of the exposure (by, for example, chemical tests) and/or the presence of symptoms of organophosphate exposure. The cardinal ocular signs of OP poisoning are pupillary shrinkage (miosis), impaired visual function and lacrimation; all of these physiologic outcomes a direct result of increased concentration of acetylcholine in the synaptic cleft of the neuromuscular junction within the ocular tissue.

The duration of exposure to OPs can be assessed using biomarkers to determine the AChE levels. At low doses OP agents induce visual incapacitation. Topical OP ocular exposure leads to a reduction in cholinesterase activity in the iris and ciliary muscle with subsequent elevation of acetylcholine in the anterior chamber fluid of the eye. This cholinergic over-stimulation action on the pupillary sphincter results in a marked miosis with dimming of vision, decrease in spatial visual ability with paralysis of the ciliary body and decreased lens accommodation, and a decline in adapting to illumination changes due to desensitization of pupillary muscarinic receptors (Egoz 2017; Dabisch 2008; Genovese 2008; Lund-Karlsen and Fonneum 1976; Takayanagi 1993). In addition, the cholinergic over stimulation action induces ciliary muscle spasm which may lead to blurred vision and myopia (Cannard, 2006; Nohara and Segawa, 1996; Rengstorff 1985; Smith and Smith 1980; Yanagisawa, 2006).

In other embodiments, the subject is at risk for organophosphate exposure. Evidence of such a risk can include, detection of the presence of organophosphates, use of organophosphates, a threatened exposure to organophosphates, the presence of symptoms of organophosphates exposure, wherein the others are in close proximity of the subject. Close proximity can be, for example, less than 1 mile, 1,500 feet, 500 feet, 100 feet, 50 feet, 25 feet, 10 feet, or 3 feet.

The composition may be administered alone or in combination with other treatments, either simultaneously or sequentially dependent upon the condition to be treated. The compositions provided herein may be dissolved in, suspended in, or admixed with one or more other active or inert ingredients. In certain embodiments, the composition can be formulated as a food item, capsule, pill, drinkable liquid, lotion, cream, emulsion, ointment, oil, gel, serum, mist, vapor, and/or combinations thereof. The compositions may also be presented in a liposome or other microparticle.

The compositions provided herein may also include other pharmaceutically-acceptable ingredients known to those skilled in the art, including, but not limited to, carriers, adjuvants, excipients, diluents, fillers, buffers, preservatives, anti-oxidants, lubricants, stabilizers, solubilizers, surfactants (e.g., wetting agents), masking agents, coloring agents and others as described below. The formulations may further comprise other active agents including, for example, other therapeutic or prophylactic agents.

As provided herein, “pharmaceutically-acceptable” refers to approved or approvable by a regulatory agency of the US Federal Government or a state government or listed in the U.S. Pharmacopeia or other generally recognized pharmacopeia for use in animals, including humans.

As used herein, “dermatologically-acceptable,” and “topically-acceptable” are used interchangeably and are intended to mean that a particular component is safe and non-toxic for application to the integument (e.g., skin) at the levels employed. In one embodiment, the components of the composition are recognized as being Generally Regarded as Safe (GRAS).

“Pharmaceutically-acceptable” carriers or adjuvants are those that can be administered to a subject, together with an active ingredient, that do not destroy the pharmacological or cosmetic activity, respectively, thereof, and which are nontoxic when administered in doses sufficient to deliver a therapeutic or cosmetic amount of the compositions provided herein. As used herein, “carrier” includes excipients.

Carriers and/or adjuvants can comprise substances used for administrating the composition according to specific routes, including, for example, oral administration, injection (e.g., subcutaneous, intravenous, intramuscular, intraperitoneal, intrathecal, and/or subdermal), and/or topical administration (e.g., via dermal absorption).

Carriers, according the subject invention, can include any and all solvents, diluents, buffers (such as neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol), and the like. The use of carriers and/or excipients in the field of drugs, cosmetics and supplements is well known.

In one embodiment, the method is used to treat a dermatological disorder, and/or to enhance the health of skin that has, or is in danger of exposure to an organophosphate.

In preferred embodiments, the compositions are formulated for topical administration, particularly for use or application to, or on, the skin. As used herein, “topical” means suitable for local application externally to the skin, or cutaneous application. In other words, a topical composition is not intended for application to a subject via oral, intravenous, intramuscular, intrathecal, subcutaneous, sublingual, buccal, rectal, vaginal, inhalation, ocular or otic routes.

Formulations suitable for topical, dermal and/or transdermal administration include, but are not limited to, gels, pastes, ointments, creams, lotions, oils, patches, adhesive plasters, bandages, dressings, depots, cements, glues, reservoirs, rinses, sprays, drops, foams, powders, sponges, tapes, vapors, tincture, and transdermal patches.

Ointments are typically prepared from the cosmetic compositions provided herein and a paraffinic or a water-miscible ointment base.

Creams are typically prepared from the cosmetic compositions provided herein and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol, polyethylene glycol, and mixtures thereof. As would be readily appreciated by one skilled in the art, formulations according to the subject invention could also comprise other alcohols, such as, for example, isopropyl alcohol or ethanol, and could also cover other alcohol based formulations, for example alcohol-based hand sanitizers.

The topical formulations may include a compound that enhances absorption or penetration of the active compound through the skin or other affected areas. Examples of such dermal penetration enhancers include dimethylsulfoxide and related analogs.

Suitable emulgents and emulsion stabilizers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the active compound in most oils likely to be used in pharmaceutical emulsion formulations may be very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Additional topical components can include, for example, emollients, such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants, such as glycerin, propylene glycol, polyethylene glycol, sorbitol solution, and 1,2,6 hexanetriol or permeation enhancers, such as ethanol, isopropyl alcohol, or oleic acid.

In some embodiments, the compositions are formulated for administration via other non-topical routes, such as, for example, via oral, intravenous, intramuscular, intrathecal, subcutaneous, sublingual, buccal, rectal, vaginal, inhalation, ocular and/or otic routes. Administration can be systemic and/or it can be local.

In one embodiment, the subject composition is formulated as an orally-consumable product, such as a food item, capsule, pill, or drinkable liquid. An orally deliverable health-promoting compound is any physiologically active substance delivered via initial absorption in the gastrointestinal tract or into the mucus membranes of the mouth.

The composition can also be formulated to be administered via, for example, injection, which includes intravenously, intraperitoneally, intramuscularly, intrathecally, or subcutaneously. The compositions can also be administered sublingually, buccally, rectally, or vaginally. Furthermore, the compositions can be sprayed into the nose for absorption through the nasal membrane, nebulized, inhaled via the mouth or nose, or administered in the eye or ear.

Formulations can include, for example, orally-consumable products, emulsions, tablets, capsules, powders, foams, granules, solutions, swabs, drops, suspensions, suppositories, injections, inhalants, and aerosols.

Orally-consumable products, according to the invention, are any preparations or compositions suitable for consumption, for nutrition, for oral hygiene, or for pleasure and are products intended to be introduced into the human or animal oral cavity, to remain there for a certain period of time, and then either to be swallowed (e.g., food ready for consumption or pills) or to be removed from the oral cavity again (e.g., chewing gums or products of oral hygiene or medical mouth washes).

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state. The orally-consumable products, according to the invention, also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance (e.g., a Qi611S Protein). In one embodiment, the orally consumable product can comprise an orally deliverable substance in powder form, which can be mixed with water or another liquid to produce a drinkable orally consumable product.

In one embodiment, the composition can be made into aerosol formulations so that, for example, it can be nebulized or inhaled. Suitable pharmaceutical formulations for administration in the form of aerosols or sprays are, for example, solutions, suspensions, or emulsions. Formulations for oral or nasal aerosol or inhalation administration may also be formulated with illustrative carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600, and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance is an acceptable isotonic solution, such as 5% dextrose or 0.9% sodium chloride; or 0.01-0.2% dipalmitoyl diphosphatidylcholine in USP WFI and 1 to 10% squalene or parenteral vegetable oil-in-water emulsions.

Additional formulations envisioned for administration to a subject include ear drops and eye drops, for treating, e.g., infections of the ears and/or eyes, and/or dry eye.

For ophthalmic use, exemplary formulations to be combined with Qi611S Protein are as follows:

### Isotonic Sodium Chloride Solution:

Sodium Chloride 0.9%
Benzalkonium chloride 1:100,000
sterile water

### Buffer Solution

boric acid 1.9%
Benzalkonium chloride 1:100,000
Sodium sulfite, anhydrous 0.1%
Phenylmercuric nitrate 1:50,000

### Artificial Tear Solution:

Polyvinyl alcohol 1.5%
povidone 0.5%
Chlorobutanol 0.5%
Ethylenediamintetaacetic acid 0.01%
Disodium edetate 0.05%
white petrolatum 55%
mineral oil 41%
lanolin 2%
thimerosal 0.002%
ammonium acetate 0.0077%
Human albumin 0.1%
   Vehicles: average drop is 25 to 50 microliters

Further components can be added to the compositions described herein as determined by the skilled artisan, for example, buffers, carriers, viscosity modifiers, preservatives, flavorings, dyes, and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

All patents, patent applications, provisional applications, and publications referred to or cited herein are incorporated by reference in their entirety, including all figures and tables, to the extent they are not inconsistent with the explicit teachings of this specification.

### EXAMPLES

It should be understood that the examples and embodiments described herein are for illustrative purposes only and that various modifications or changes in light thereof will be suggested to persons skilled in the art and are to be included within the spirit and purview of this application.

### Example 1—ISOLATION AND TRANSFORMATION OF Qi611S GENE INTO E. coli BL21

A gene encoding Qi611S, Qi611S, can be isolated from Lactobacillus fermentum Qi6 and transformed into E. coli BL21. As shown in FIG. 3, E. coli BL21 is capable of synthesizing a Qi611 S protein.

Primers that were used to amplify Qi611S from LF Qi6 can be used to confirm the presence of Qi611S in the E. coli cell. The primers used for cloning of Qi611S into the pET-15b vector are listed in Table 2 (see also FIGS. 1-2). These specially-designed primers were obtained from Integrated DNA Technologies (IDT) (Coralville, Iowa).

TABLE 2
Primers used in the cloning of Qi611S for
transformation into E. coli BL21.

| Primer Name | Primer Sequence (5′-3′) |
|---|---|
| Lacto F | CATATGGATAACCGGATTTTCTTCA (SEQ ID |
| | NO: 3) |
| Lacto R | GGATCCTTAGAGGTTTTTCTTAATC (SEQ ID |
| | NO: 4) |

DNA was isolated and amplified from the Lf Qi6 chromosome that encodes Qi611S using the primers featured in Table 2. The primers enabled the use of two restriction enzymes, NdeI and BamHI, for a restriction enzyme digest of the amplified Qi611S gene and the multiple cloning site of the pET-15b vector.

Once the amplified DNA and the vector are digested using the restriction enzymes, the Qi611S gene is ligated into the vector. The vector can then be transformed into E. coli BL21.

The transformed E. coli culture is grown overnight at 37° C. and individual colonies are tested for the presence of Qi611S using PCR. Once positive clones are identified, the bacteria can then be grown to identify if Qi611S is successfully encoding synthesis of Qi611S.

The Qi611S protein can be detected by silver staining on SDS-PAGE gel. The SDS-PAGE gel is loaded with released protein samples from E. coli BL21 that is expressing Qi611S. The pET-15b vector encodes a polyhistidine-tag, enabling the identification of a synthesized protein without having an antibody specific to the Qi611S. Additionally, the pET-15b encodes the thrombin protease cleavage site (Leu-Val-Pro-Arg-Gly-Ser) to enable removal of the polyhistidine-tag.

### Example 2—Assay for Testing Effects of Ocular Organophosphate Toxicity Induced by Exposure to Paraoxon

Paraoxon, an organophosphorus compound that acts as an irreversible AchE inhibitor and may be used as a surrogate for military-grade nerve agents such as sarin, VX, or novichok, was used to study organophosphate toxicity in an ex vivo rabbit eye model. The study involved the use of compound Qi611 to evaluate its effectiveness for protection of ocular AChE against paraoxon-induced inhibition, and activation of ocular peroxisome proliferator-activated receptor PPAR-γ.

### Materials and Methods

### 1. AChE Inhibitor Challenge of Rabbit Eyes

Whole ex vivo eyes with conjunctiva from young (8- to 12-weeks old) New Zealand white rabbits used in this study were purchased from Pei-Freez, LLC (Rogers, AR, USA). Custom organ collection enabled one eye from the same animal to serve as a control (e.g., saline or DMSO) with the other eye being subject to 611 prophylactic treatments followed by paraoxon challenge. That setup facilitated the limitation of possible confounding factors such as discrepancies in the age of animals, gender bias, or genetic differences related to animals used in the study, which collectively could influence tissue susceptibility to OP challenge. At least six eyes were assigned to each treatment group.

The eyes were transported on wet ice in a drug- and serum-free DMEM cell culture medium and kept at 4° C. until ready for processing. Paraoxon-ethyl (pOX) was purchased from Sigma-Aldrich (St. Louis, MO, USA) with aliquots prepared fresh before each experiment due to the limited stability of OP compounds. Dimethyl sulfoxide (DMSO) and sterile saline were used for the preparation of serial dilutions of paraoxon-ethyl used in the experiments, and all experiments were conducted under a laminar flow hood to reduce risks of contamination. Compound 611S was resuspended in sterile saline.

Briefly, rabbit eyes were removed from the transport medium and transferred into 3 mL of fresh drug-free DMEM-F12 Ham's 1:1 culture medium with one eye per well being placed into a 6-well tissue culture plate. The eyes were permitted to warm up to 20° C. (usually in approximately 15 min) before any experimental procedures were carried out, with temperature measurements conducted with a no-touch infrared thermometer (Braun BNT100CN, Kaz USA, Inc.). Surgical forceps were used for pulling back eyelids to expose eye orbits followed by gentle rinsing of corneas with 1 mL of drug- and serum-free DMEM-F12 culture medium. Sterile nylon rings (ϕ 15 mm) were gently fitted around the corneal-limbal area to contain saline, DMSO, or 611 treatments (volumes of 200 μL per eye). Treatment solutions were left on the corneal surface for 10 min followed by ring removal to drain the treatment solutions. After draining, fresh sterile nylon rings were placed around the corneal area and paraoxon was dispensed directly onto the corneal surface as 20 μL droplets, with three droplets total dispensed 5 min apart to allow for tissue penetration of paraoxon solutions to ocular tissues.

Pupil sizes were monitored for 40 minutes from dispensing of the first dose of paraoxon, with pictures taken every five minutes. Digital pictures of the pupils were used for subsequent ImageJ (NIH, USA) analyses of changes in sizes of the pupillary area over time, in addition to physical measurements of pupillary diameters with an electronic caliper (digital caliper 15-719-8, Swiss Precision Instruments, was used in the study).

After paraoxon challenge treatments, anterior chamber fluid was drawn from rabbit eyes with a hubless U-100 insulin syringe and a 31 G needle, and the fluid samples dispensed into Eppendorf tubes were immediately placed in −80° C. until they were needed for further analyses. The amount of anterior chamber fluid was between 100-250 μl, depending on eye sizes.

### 2. AChE Enzymatic Activity Colorimetric Test

Acetylcholinesterase assay colorimetric kit ab138871 (Abcam, USA) was used for measuring AChE activity in the anterior chamber fluid samples. The assay followed the manufacturer's recommendations included in the kit. Acetylcholinesterase standard (1 mU/ml to 1,000 mU/ml) replicates, experimental samples (5 μL at indicted concentrations added to 45 μL reaction buffer), and blank controls (assay buffer only) are added in 50 μL total volumes to indicated microplate wells. Paraoxon (5 μL added to 45 μL reaction buffer) controls and standard curve were also applied to validate the model. Samples are incubated for 10 minutes, followed by the addition of 50 μL of the acetylthiocholine reaction mix to each well, and incubated for 30 minutes at room temperature protected from light. Absorbance increase at 410 nm is analyzed with a microplate reader. Corrected readings are obtained by subtracting background absorbance (blank wells), and trendline equation generated from the standard curve.

### 3. PPAR-γ Levels in Ocular Tissues

Measurements of peroxisome proliferator-activated receptor-gamma upregulation in the rabbit eyes in response to paraoxon challenge used immunoblotting with anti-PPAR-γ mAb (Abcam, USA). Briefly, anterior fluid samples have been resolved with SDS-PAGE on NuPAGE 4-12% Bis-Tris midi-protein gels (Invitrogen, USA) followed by transferring of proteins onto a PVDF Immobilon-P membrane (Millipore, USA). The membranes were blocked overnight at 4° C. in 20 mL of the PBS-T buffer with 5% w/v non-fat dry milk (Publix, USA). After blocking, the membranes were incubated with a mouse monoclonal anti-PONI or with anti-PPARy antibody (1:5,000 titer) for two hours with gentle rocking, followed by three five-minute-long rinses with PBS-T. The membranes were then placed into 20 mL of fresh PBS-T buffer with 5% w/v non-fat dry milk for one-hour incubation with the secondary anti-mouse HRP-conjugated antibody (1:10,000) and with gentle rocking. The SuperSignal Femto kit was used for immunoblotting signal development and visualization (Thermo Scientific, USA).

### 4. LDH Release Assay

Anterior chamber fluid samples were used for quantification of lactate dehydrogenase (LDH) activity. A colorimetric LDH assay kit ab102526 (Abcam, USA) was used following the manufacturer's recommended protocol with 21 of samples being used in the assay.

### 5. Statistical Analyses

One-way ANOVA with Dunnett's post hoc test, and linear regression models, were used for evaluation of paraoxon treatment effects on pupillary sizes of ex vivo rabbit eyes (maximal observed miosis) and levels of activity of AChE, PPAR-γ or LDH detected in anterior chamber fluid. Statistical significance was assumed with a p-value<0.05.

### Results

### 1. Assessment of Paraoxon-Induced Miosis in Ex Vivo Rabbit Eyes

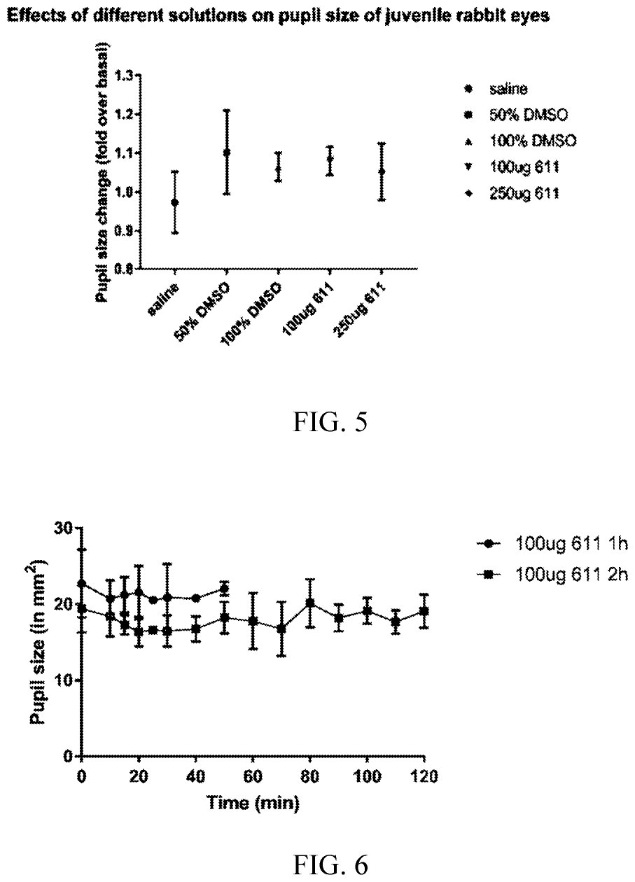

To decrease variation associated with phenotypic and genotypic differences a custom collection of eyes were used in the experiments. Composing treatment groups on the basis per animal rather than per number of eyes, use of standardized temperatures of the eye in the experiments, validation of digitally measured pupil sizes with physical measurements with a digital caliper, and work with small eye batches to limit risks associated with tissue decay over time with a designated person for every step of the experiment to limit operator error (e.g., collection of anterior chamber fluid) contributed to reproducible results of the study. Comparison of pupil sizes recorded after 40 minutes (t40) since adding the first drop of paraoxon (t0, basal value) did not show significant differences between eyes treated with saline, DMSO, or 611 (FIG. 5).

FIG. 5. Saline, DMSO, or 611 alone do not significantly affect pupil sizes of ex vivo rabbit eyes.

Additionally, saline preparation of Qi611S did not cause significant changes in pupil sizes even within two hours after 10 min pretreatment with Qi611 S, demonstrating that Qi611 S alone does not act as a miotic nor a mydriatic agent (FIG. 6).

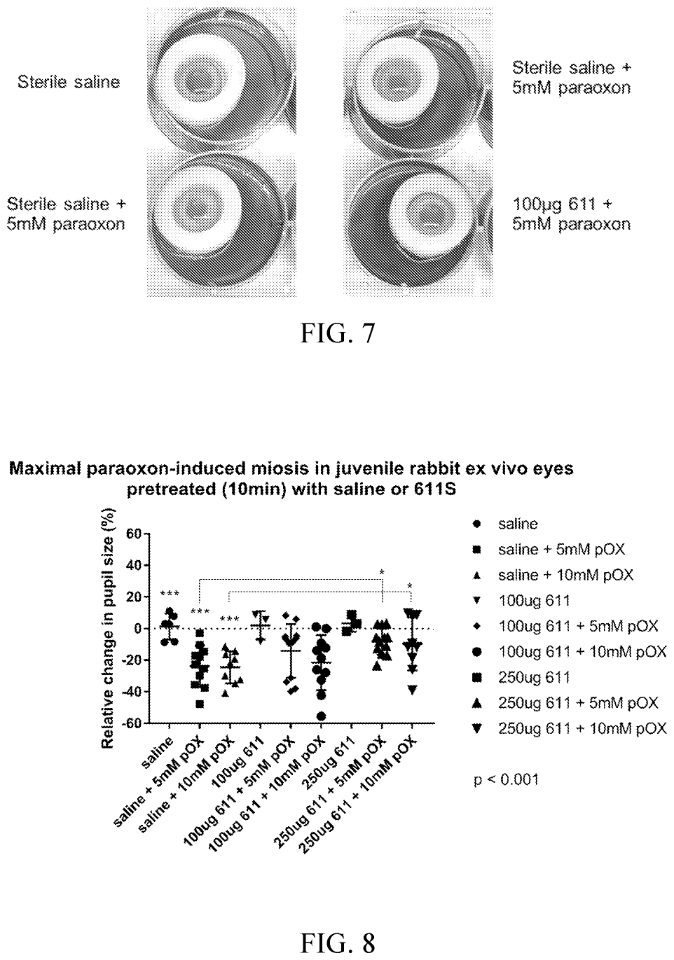

Eyes subject to paraoxon treatment at a concentration of 5 mM or 10 mM exhibited significant miosis with a considerable reduction in pupil sizes when compared to eyes treated only with saline or 611 (p<0.005). Additionally, ex vivo rabbit eyes exposed to 250 μg dose of 611 for 10 min and later challenged with 5 mM or 10 mM paraoxon, shown significantly weaker miosis when compared to eyes challenged with paraoxon that were pretreated for 10 min with saline (FIG. 7, FIG. 8). This attenuation of OP-induced miosis correlates with the protection of AChE activity and upregulation of PPAR-γ.

### 2. AChE Enzymatic Activity—Colorimetric Test Data

Anterior chamber fluid collected from eyes treated with Qi611 S and exposed to paraoxon shows significantly higher AChE activity, while eyes treated with saline show approximately 80% reduction of the enzymatic activity, consistent with inhibition of AChE typically seen after the addition of OP compounds (FIG. 9). Depending on paraoxon concentration, 100 μg of 611 can preserve 50 to 80% of enzymatic activity of acetylcholinesterase.

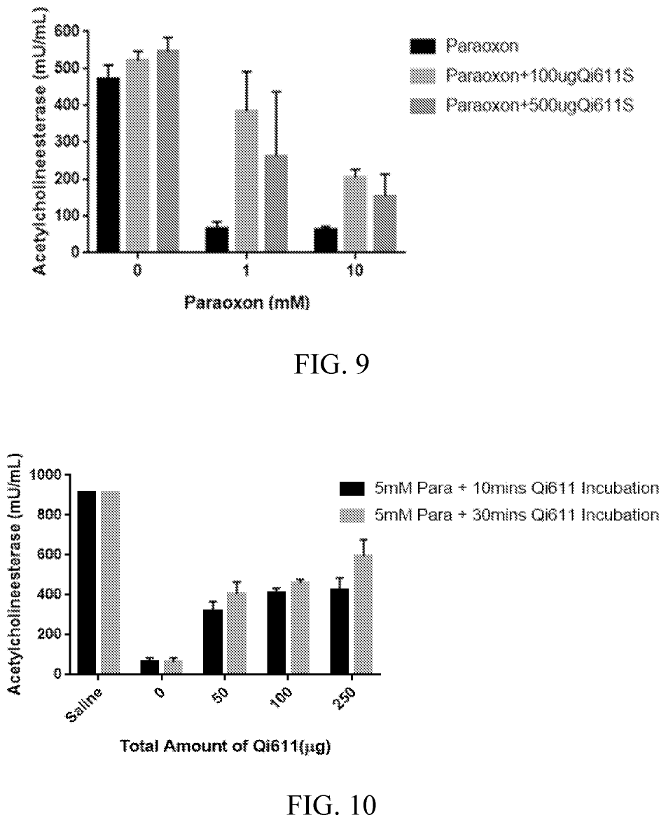

FIG. 10. Protection of AChE activity by Qi611S is rapid as demonstrated with 10- and 30-min prophylactic treatment before paraoxon exposure.

### 3. PPARy-γ Activity—Western Blotting

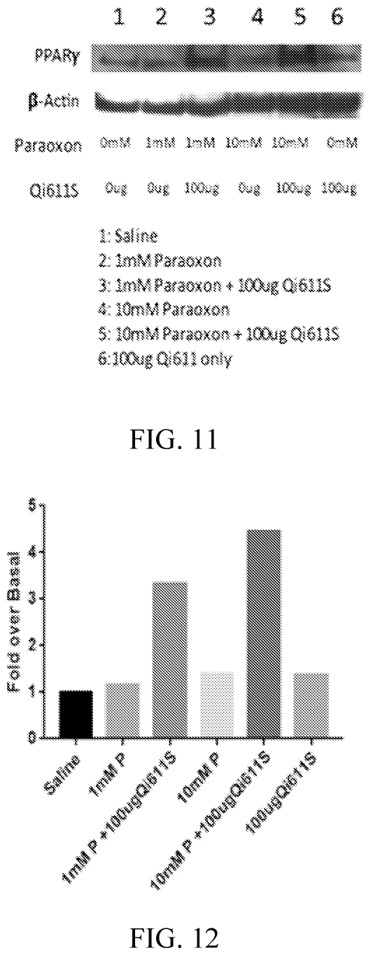

Eyes treated with Qi611S before paraoxon exposure, exhibited three- to four-fold higher upregulation of PPAR-γ as demonstrated with immunoblotting (FIG. 11). These results were consistent when using eyes from different cohorts of rabbits indicating Qi611S′ role as a conditional PPAR-7 agonist. Upregulation of PPAR-γ correlates with the protection of AChE activity in ex vivo rabbit eyes treated with Qi611S. Paraoxon (P) at 1 mM and 10 mM concentrations were used in this experiment.

FIG. 12 shows that prophylactic treatment with Qi611S before paraoxon exposure decreases tissue damage as demonstrated with LDH release assay.

### Example 3—Biofilm Production

### Culture of Lf Qi6 Biofilm

After being isolated and identified, Lf Qi6 was cultured in MRS agar plate. The culture was then incubated in 5 ml of MRS broth for 24 hours at 37° C. 1 ml of the culture was transferred into a T-150 tissue culture plate with 25 ml of MRS broth. 25 ml of MRS media was changed every 48 hours to allow the biofilm of Lf Qi6 to grow as lawn on the bottom of the culture plate. The culture was then grown for 7 days to produce a thick biofilm layer. The grown biofilm layer was subsequently scraped out and suspended in fresh medium. Freezer stacks were made with glycerol and stored in −80°.

A biofilm phenotype of Lf Qi6 in frozen stock was cultured in 10 ml of fresh MRS media for 24 hours at 37°. 10 ml of culture was inoculated into 25 L of MRS media with 500 g sterile glass wool. The biofilm was then cultured for 72 hours under static conditions at 37° C. The culture was mixed every 24 hours with a gentle shaking, after which the media and glass wool were harvested.

The biofilm cells were subsequently detached from the glass wool via sonication. The cells were further centrifuged to concentrate the biofilm of Lf Qi6, which was then suspended in sterile water. This scale-up yields a biofilm culture at a concentration of 50 g/25 L. The Lf Qi6 biofilm growth is further illustrated in FIG. 4, wherein the biofilm was cultured on substrates in a scaled-up culture as described herein.

### Lf Qi6 Biofilm Downstream Processing

50 g of biofilm phenotype of Lf Qi6 was suspended in 1 L of sterile water. The suspension was gently mixed for 24 hours at room temperature to allow the passive release of multiple bioactives. The mixture was then sonicated for 30 minutes (50 KHz, 200 watt) into uniform lysate using an OmniSonic Ruptor 400. The sonicated lysate was then frozen and lyophilized into a fine powder.

### REFERENCES

1. Centers for Disease Control and Prevention, (1984) Epidemiologic notes and reports—Organophosphate insecticide poisoning among siblings—Mississippi. Morbidity and Mortality Weekly Report, Oct. 26, 1984, 33(42): 592-4
2. Lin T J, Walter F G, Hung D Z, Tsai J L, Hu S C, Chang J S, Deng J-F, Chase P B, Denninghoff K, and Chan H M (2008) Epidemiology of organophosphate pesticide poisoning in Taiwan, Clin Toxicol, 46:9, 794-801
3. Meulenbelt S E and Nieuwenhuizen M S., (2015) Non-state actors' pursuit of CBRN weapons: From motivation to potential humanitarian consequences. IRRC 97(899): 831-858
4. Mew E J, Padmanathan P, Konradsen F, Eddleston M, Chang S-S, Phillips M R, and Gunnell D., (2017) The global burden of fatal self-poisoning with pesticides 2006-2015: Systematic review. J Affect Disord 219: 93-104
5. Bertolote J M, Fleischmann A, Eddleston M, and Gunnell D., (2006) Deaths from pesticide poisoning: a global response. Br J Psychiatry 189: 201-203
6. Organization for the Prohibition of Chemical Weapons., (2018) Note by the OPCW Technical Secretariat S/1671/2018 related to the “Amesbury Incident” https://www.opew.org/sites/default/files/documents/2018/09/s-1671-2018%28e %29.pdf
7. John H, van der Schans M J, Koller M, Spruit H E T, Worek F, Thiermann H, and Noort D., (2018) Fatal sarin poisoning in Syria 2013: forensic verification within an international laboratory network. Forensic Toxicol 36(1): 61-71
8. Steindl D., Boehmerle W, Korner R, Praueger D, Haug M, Nee J et al., (2020) Novichok nerve agent poisoning. Lancet 397(10270): 249-252
9. Posno M, Leer R J, van Luijk et al. Incompatibility of Lactobacillus Vectors with Replicons Derived from Small Cryptic Lactobacillus Plasmids and Segregational Instability of the Introduced Vectors. Applied and Environmental Microbiology 1991: 57(6): 1822-1828.
10. Felten A, Grandry B, Lagrange P H et al. Evaluation of Three Techniques for Detection of Low-Level Methicillin-Resistant Staphylococcus aureus (MRSA): a Disk Diffusion Method with Cefoxitin and Moxalactam, the Vitek 2 System, and the MRSA-Screen Latex Agglutination Test. J Clin Microbiol 2002: 40 (8): 2766-2771.
11. Mukherjee, S., Gupta, R. D., Organophosphorus Nerve Agents: Types, Toxicity, and Treatments, J Toxicol., 2020, 2020: 3007984.

We claim:

1. A method for alleviating toxicity caused by a toxin wherein said method comprises administering, to a subject in need of alleviation of such toxicity, a protein that comprises an amino acid sequence selected from:
i) SEQ ID NO: 1;
ii) a variant of SEQ ID NO: 1; and
iii) a fragment of i) or ii) wherein said variant or fragment reduces toxicity.

2. The method of claim 1, wherein the protein comprises SEQ ID NO: 1, or is a variant of SEQ ID NO: 1 having at least 75% sequence identity to SEQ ID NO: 1, or is a fragment of SEQ ID NO: 1 or a fragment of said variant, wherein said fragment has a length of at least 20% the length of SEQ ID NO: 1.

3. The method of claim 1, wherein the protein comprises SEQ ID NO: 1.

4. The method of claim 1, wherein the protein has SEQ ID NO: 1.

5. The method of claim 1, wherein the protein comprises a fragment of at least 70% of SEQ ID NO: 1.

6. The method of claim 1, wherein the toxin is an organophosphate.

7. The method of claim 6, wherein the organophosphate is a pesticide.

8. The method of claim 6, wherein the organophosphate is a nerve agent.

9. The method of claim 1, wherein the toxin is a tetrodotoxin (TTX).

10. A method for assessing toxicity caused by an organophosphate wherein said assay comprises administering an organophosphate to a rabbit eye ex vivo an organophosphate and measuring, in the rabbit eye, miosis and AChE enzymatic activity, wherein the presence of miosis and a reduction of AChE enzymatic activity indicates organophosphate toxicity.

11. The method of claim 10, used to evaluate the ability of a candidate composition to reduce organophosphate toxicity wherein the method comprises the use of two ex vivo rabbit eyes wherein one of said eyes serves as a control and receives an administration of the organophosphate, but not the candidate composition, and the other eye serves as a test receives both the candidate composition and the organophosphate, wherein the extent of miosis and AChE activity in each eye is measured and compared to each other, and wherein less miosis and more AChE activity in the test eye indicates that the candidate compound reduces organophosphate toxicity.

12. A method for alleviating toxicity caused by a toxin wherein said method comprises administering, to a subject in need of alleviation of such toxicity, a composition that comprises a Lactobacillus fermentum bacterium grown in a biofilm phenotype and/or an extract from said L. fermentum and/or said biofilm.

13. The method of claim 12, wherein the toxin is an organophosphate.

14. The method of claim 13, wherein the organophosphate is a pesticide.

15. The method of claim 13, wherein the organophosphate is a nerve agent.

16. The method of claim 12, wherein the toxin is a tetrodotoxin (TTX).

17. The method of claim 12, wherein the L. fermentum is LFQi6 (ATCC No. PTA-122195).


SEQUENCE LISTING (6 sequences)

SEQ ID NO: 1 - protein, 74 aa, Lactobacillus fermentum
MDNRIFFNPGDSIANIHDYNEAVRKGQIFKKEQQAGDLVIAKGPDDEEYAIFYANDALPADHEQSQPYEIKKNL

SEQ ID NO: 2 - DNA, 225 bp, Lactobacillus fermentum
  source 1..225: mol_type = genomic DNA
atggataaccggattttcttcaaccccggcgactcgatcgccaacatccacgactacaacgaagccgtccgcaagggccaaatcttcaaaaaggaacagcaggccggcgacctcgtgatcgctaagggtcccgatgacgaagaatacgccatcttctacgccaacgatgccctgcccgccgaccacgagcaatcccaaccctacgagattaagaaaaacctctaa

SEQ ID NO: 3 - DNA, 25 bp, Lactobacillus fermentum
  source 1..25: mol_type = genomic DNA
catatggataaccggattttcttca

SEQ ID NO: 4 - DNA, 25 bp, Lactobacillus fermentum
  source 1..25: mol_type = genomic DNA
ggatccttagaggtttttcttaatc

SEQ ID NO: 5 - DNA, 287 bp, synthetic construct
  misc_feature 1..287: note = primer
  source 1..287: mol_type = other DNA
agatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgctcgaggatccggctgctaacaaagcccgaaaggaagctgagttggctgctgccaccgctgagcaataactagcataaccccttggggcctctaaacgggtcttgaggggttttttg

SEQ ID NO: 6 - protein, 43 aa, synthetic construct
  REGION 1..43: note = primer
MGSSHHHHHHSSGLVPRGSHMLEDPAANKARKEAELAAATAEQ